(19) United States
(12) Patent Application Publication
(10) Pub. No.: US 2022/0000164 A1
(43) Pub. Date: Jan. 6, 2022

(54) NUTRITIONAL COMPOSITIONS, THEIR USE IN REDUCING METABOLIC STRESS AND METHOD OF REDUCING METABOLIC STRESS

(71) Applicant: SOCIETE DES PRODUiTS NESTLE S.A., Vevey (CH)

(72) Inventors: Jalil Benyacoub, Epalinges (CH); Nicholas Paul Hays, La Tour-De-Peilz (CH)

(21) Appl. No.: 17/289,016

(22) PCT Filed: Oct. 30, 2019
(86) PCT No.: PCT/EP2019/079650

§ 371 Date: Apr. 27, 2021

(51) Int. Cl. A23L 33/00; A23L 33/155; A23L 33/19; A23L 33/12; A61P 3/00; A61K 31/202

(52) U.S. Cl. CPC A23L 33/30; A23L 33/155; A61K 31/202; A23L 33/12; A61P 3/00; A23L 33/19

(57) ABSTRACT

The present invention concerns a nutritional composition, for example a synthetic nutritional composition, for use in reducing the metabolic stress. This composition is for use in mammals, preferably in humans, more preferably in infants. The nutritional composition comprises one or more medium chain fatty acid (MCFA) derivative or derivatives for use in reducing metabolic stress. Conditions and/or diseases associated to the metabolic disorders and/or imbalances in an infant are selected in the group consisting of neurological, growth and/or gut retarded development or abnormalities, hypoglycemia, hyperglycemia, hyperinsulinemia, hypertriglyceridemia. The infant is born preterm or with low-birthweight (LBW) or who experienced intra-uterine growth retardation (IUGR) or small for gestational age (SGA)).

### FIELD OF THE INVENTION

The present invention concerns a nutritional composition, for example a synthetic nutritional composition, for use in reducing the metabolic stress. This composition is for use in mammals, preferably in humans, more preferably in infants.

### BACKGROUND OF THE INVENTION

Post-natal microbial colonization plays a major role in educating/imprinting infant metabolic digestive and immune functions. It is also well reported that post-natal type of feeding significantly influence the microbiota balance and the physiological functions, including metabolism and immunity and overall growth.

It is assumed that some specifically adapted nutritional solutions (formulas or fortifiers) may reduce metabolic stress and be therefore more appropriate for the status of gut development at this early life stage. These can promote growth and prevent metabolic disorders.

Elastase is a proteolytic pancreatic enzyme. Series of reports have investigated fecal elastase levels in infant stool samples as marker of pancreatic function (Nissler et al. 2001; David-Henriau et al. 2005). Elastase has also been documented as a human milk component (Borulf et al. 1987). The adult reference value for fecal elastase is around 200 μg/g of stool. In newborns, the fecal elastase level increase during the first two weeks of life and then is rather maintained at stable adult-like levels after the weaning (Nissler et al. 2001). Conclusions of most of the studies mentioned above indicated that normal fecal elastase levels are reached around day 3 in term newborns.

Positive nutritional impact of early feeding on fecal elastase levels was evidenced in pre-term babies as compared to reference term babies (Jurges et al. 1996; Campeotto et al. 2002).

Preterm babies exhibit pancreatic immaturity and high sensitivity to environmental stress, including inappropriate nutrient up-take related to gut barrier defect, that may lead to increased elastase production and secretion into the gut lumen. Indeed, such elevated levels of elastase, a sub-family member of serine proteases, may in turn affect gut barrier lining and function by consequence alter nutrient absorption and as well may trigger inflammation and impact organ functions, incl. kidney, liver and pancreas thus contributing to the metabolic stress to which such premature babies are exposed. It is in fact hypothesized that altered gut barrier will lead to “excessive” passage of nutrients and food antigens that will bring a challenge to pancreas, with consequent further production of pancreatic elastase, and create a vicious circle that will lead in critical conditions to organ dysfunctions. This is particularly relevant to premature infants who are fragile and exposed to additional risks such as NEC and sepsis.

None of these prior art documents addresses anyway the issue of reducing the metabolic stress in infants that may happen for example by the introduction of infant formula, and that may affect the gut permeability. There is also no focus on providing a nutritional composition which promotes a rate of growth of the infant that is closer to the one obtained for breast-fed infants.

Accordingly, there is a need to find ways to reduce the metabolic stress in infants, in particular in infant who are born pre-term or with low-birth weight (LBW) or experienced intra-uterine growth retardation (IUGR), such as suboptimal intra-uterine nutrition, and/or disease.

There is more generally a need for nutritional intervention achieving the above mentioned benefits in young mammals, in particular infants and children, preferably infants, but also young pets.

### SUMMARY OF THE INVENTION

The present invention relates to a nutritional composition, for example a synthetic nutritional composition, for infants, in particular for infants born preterm or with low-birth weight (LBW) or who experienced intra-uterine growth retardation (IUGR) or small for gestational age (SGA), such as a pre-term formula or a human milk fortifier. The composition comprises medium chain fatty acid (MCFA) derivatives. The compositions according to the invention have been surprisingly found to generate reduced levels of fecal pancreatic elastase with respect to control compositions not comprising MCFA derivatives.

In the study presented in this application, pre-term infants fed with human milk fortified with a human milk fortifier according to the present invention presented normalized levels of faecal elastase which were also lower than the control groups fed with human milk fortified with a standard fortifier.

As explained in the background of the invention increased pancreatic elastase secretion is known to be an indicator (marker) of metabolic stress and the observed trend in its reduction indicates that the compositions of the invention contribute to a positive effect on homeostasis and may dampen a stress on metabolic functions for the infant, in particular an infant born preterm or with low-birth weight (LBW) or who experienced intra-uterine growth retardation (IUGR) or small for gestational age (SGA), who is fed with the composition.

Accordingly, in one aspect, the present invention provides a nutritional composition, for example the synthetic nutritional composition, comprising one or more medium chain fatty acids (MCFA) derivative for use in reducing the metabolic stress in infants, for example in infants who were born pre-term or with low-birth weight (LBW) or experienced intra-uterine growth retardation (IUGR).

In another aspect, the present invention provides the use of one or more MCFA derivative for the manufacture of a nutritional composition, for example the synthetic nutritional composition, for reducing the metabolic stress in infants, for example in infants who were born pre-term or with low-birth weight (LBW) or experienced intra-uterine growth retardation (IUGR).

In yet another aspect, the present invention provides for a method for reducing the metabolic stress in infants in need thereof, for example in infants who were born pre-term or with low-birth weight (LBW) or experienced intra-uterine growth retardation (IUGR) comprising administering to such subject a nutritional composition, for example the synthetic nutritional composition, comprising one or more medium chain fatty acids

(MCFA) derivative.

In another aspect, the present invention provides for the use of a nutritional composition, for example a synthetic nutritional composition, comprising one or more medium chain fatty acids (MCFA) derivative to reduce the metabolic stress in infants, for example in infants who were born pre-term or with low-birth weight (LBW) or experienced intra-uterine growth retardation (IUGR).

### BRIEF DESCRIPTION OF THE DRAWINGS

Additional features and advantages of the present invention are described in, and will be apparent from, the description of the presently preferred embodiments which are set out below with reference to the drawings in which:

FIG. 1 shows the geometric mean concentration of fecal elastase-1 as described in Example 2. As shown, levels of fecal elastase were significantly lower in the nHMF group compared to the cHMF group at D21. In addition, the increase from FSI1 to D21 was significantly less in the nHMF group compared to the cHMF group.

### DETAILED DESCRIPTION OF THE INVENTION

Definitions

As used herein, the following terms have the following meanings.

The term “subject” as used herein refers to a mammal, in particular a cat, dog or human, more particularly the term refers to a human, even more particularly a human infant or child and even more particularly still a human infant or child fed infant formula and/or growing up milk.

The term “infant” as used herein refers to a human infant of up to 12 months of age and includes preterm and very preterm born infants, infants having a low birth weight i.e. a new born having a body weight below 2500 g (5.5 pounds) either because of preterm birth or restricted fetal growth, and infants born small for gestational age (SGA) i.e. babies with birth weights below the 10th percentile for babies of the same gestational age.

The term “child” as used herein refers to a human of 1 to 18 years of age, more specifically a human of 1 to 10 years of age, even more specifically a human of 1 to 5 years of age, and even more specifically a human of 1 to 2 years of age.

The term “formula fed infant or child” as used herein refers to an infant or child fed either infant formula and/or growing up milk.

The term “breastfed subject” as used herein refers to a subject, In particular an infant or child, fed human breastmilk, in particular from a nutritionally replete mother.

A “preterm” or “premature” means an infant or young child that was not born at term. Generally it refers to an infant born prior to the completion of 37 weeks of gestation.

The expression “Term born infant” indicates an infant born after 37 weeks gestation.

Within the context of the present invention, the term “Low birth weight” indicates a newborn's body weight below 2500 g (5.5 pounds), either as a result of preterm birth (i.e. before 37 weeks of gestation) and/or due to restricted foetal growth.

By the expression “low birth weight”, it should be understood as any body weight under 2500g at birth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

Within the context of the present invention, the term “Small-for-gestational-age (SGA)” refers to babies with birth weights below the 10th percentile for babies of the same gestational age.

The expression “Postnatal period” is the period beginning immediately after the birth of a child and extending for about six weeks.

The expression “nutritional composition” means a composition which nourishes a subject. This nutritional composition is usually to be taken enterally, orally, parenterally or intravenously, and it usually includes a lipid or fat source and optionally a protein source and /or optionally a carbohydrate source and/or optionally minerals and vitamins. Preferably, the nutritional composition is for oral use.

The expression “hypoallergenic nutritional composition” means a nutritional composition which is unlikely to cause allergic reactions.

The expression “synthetic composition” means a mixture obtained by chemical and/or biological means, which can be chemically identical to the mixture naturally occurring in mammalian milks.

The expression “synthetic nutritional composition” identifies nutritional composition as above defined which are obtained by chemical and/or biological means, which can be chemically identical to a the mixture which also naturally occurr, for example in mammalian milks. As detailed in Example 1, synthetic nutritional compositions as herein defined are comprised within the scope of the present invention and all the embodiments described in the present application apply as well to such synthetic nutritional composition.

In an embodiment, said synthetic nutritional composition is selected from the group consisting of; growing up milk, infant formula or a composition for infants that is intended to be added or diluted with human breast milk (hereinafter “HM”) e.g. HM fortifier, or a food stuff intended for consumption by an infant and/or child either alone or in combination with HM e.g. complementary foods.

The expression “infant formula” means a foodstuff intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae).

The expression “starter infant formula” means a foodstuff intended for particular nutritional use by infants during the first four months of life.

The expression “pre-term formula” or “preterm formula” means an infant formula intended for a preterm infant or for an infant with low-birth weight (LBW) or who experienced intra-uterine growth retardation (IUGR) or for infants small for gestational age (SGA).

The expression “fortifier” or “human milk fortifier” (HMF) refers to liquid or solid nutritional compositions suitable for mixing with breast milk or infant formula, for example a preterm infant formula. By the term “milk fortifier”, it is meant any composition used to fortify or supplement either human breast milk, infant formula, growing-up milk or human breast milk fortified with other nutrients. The term “fortifier” refers to a composition which comprises one or more nutrients having a nutritional benefit for infants, both preterm infants, with low-birth weight (LBW) or infants who experienced intra-uterine growth retardation (IUGR) or infants small for gestational age (SGA), and term infants.

The term “weaning period” means the period during which the mother's milk is substituted by other food in the diet of an infant.

The “mother's milk” should be understood as the breast milk or colostrum of the mother (=Human Breast Milk =HBM).

The expression “metabolic stress” should be understood as a situation during which an unforeseen physical, chemical or biological factor (insult) brutally modifies homeostasis, therefore nutrient's metabolism and nutritional needs of an individual (Colomb, V., Nutrition Clinique et Metabolisme, 2005: 19: 229-33). In the context of the present invention, the stress factor considered in this case (i.e. in infant) may be due to the change in feeding and the introduction of an infant formula containing substances that are encountered for the first time by the infant organism. The way of delivery may also be considered as a stress factor. C-section may induce stress that impacts metabolic health in the newborn. A too frequent antibiotic use early in life may also be a factor inducing a metabolic stress, as well as the fact the infant is born preterm and/or small-for-gestational age. The expression “reducing the metabolic stress” of an individual implies a reduction of the metabolic disorders and/or imbalances—especially those resulting from an unforeseen chemical, nutritional or biological factor (insult)—such as a change of homeostasis, nutrient's metabolism, nutritional needs of said individual. It also encompasses the treatment (e.g. a reduction of the occurrences/severities) of conditions and/or diseases associated to the metabolic disorders and/or imbalances, known by the skilled person. One embodiment of the present invention therefore refers to a nutritional composition as described in the present invention for use in the prevention and/or treatment of conditions and/or diseases associated to the metabolic disorders and/or imbalances in an infant, especially by reducing the metabolic stress in said infant in the first twelve months of life. Some examples of conditions and/or diseases associated to the metabolic disorders and/or imbalances include neurological, growth and/or gut retarded development or abnormalities, hypoglycemia, hyperglycemia, hyperinsulinemia, hypertriglyceridemia.

The term “fatty acid” as used herein indicates a carboxylic acid with a long aliphatic chain, which is either saturated or unsaturated and refers to a compound of formula (XII)

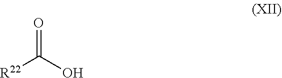

Wherein

R²² is a C3 to C43 branched or unbranched acyclic alkyl, or acyclic alkenyl group. More particularly, R²² is a C3 to C43 branched or unbranched acyclic alkyl, or acyclic alkenyl group, and even more particularly a C3 to C 28 branched or unbranched acyclic alkyl, or acyclic alkenyl group. Mixture of such compounds are also comprised within the scope of the invention and/or the term.

The term “medium chain fatty acid” (MCFA) as used within the context of the present invention identifies a fatty acid as above defined wherein R²² is C₇ or C₉ branched or unbranched acyclic alkyl, or acyclic alkenyl group. Non limiting examples of such MCFA are: capric acid (8:0) and caprylic acid (10:0). Mixture of such compounds are also comprised within the scope of the invention and/or the term.

The term “long chain fatty acid” (LCFA) as used within the context of the present invention identifies a fatty acid as above defined wherein R²² is C₁₁branched or unbranched acyclic alkyl, or acyclic alkenyl group or longer, in particular C₁₃ to C₂₃. Long chain fatty acids may be saturated, mono unsaturated (MUFA) or polyunsaturated (PUFA). Non limiting examples of such LCFA are: lauric acid (12:0), myristic acid (14:0), palmitic acid (16:0), stearic acid (18:0), arachidic acid (20:0), behenic acid (22:0), lignoceric acid (24:0), vaccenic acid (n-7, 18:1), gondoic acid (n-9, 20:1), erucic acid (n-9, 22:1), mead acid (n-9, 20:3), alpha-linolenic acid (ALA) (n-3, 18:3), Eicosapentaenoic acid (EPA) (n-3, 20:5), Docosapentaenoic acid (DPA n-3) (n-3, 22:5), Docosahexaenoic (DHA) (n-3, 22:6), Linoleic acid (LA) (n-6, 18:2), Dihomo-gamma-linolenic acid (DGLA) (n-6, 20:3),

Arachidonic acid (AA or ARA) (n-6, 20:4), and Docosapentaenoic acid (DPA n-6) (n-6, 22:5). Long chain fatty acids are typically product of fatty acid metabolism in humans.

LCFA belonging to the n-6 and n-3 series constitute the so called “essential fatty acids” whose biosynthesis can't be initiated by metabolic mechanisms in the absence of linoleic and alpha-linoleic acid substrate introduced with the diet.

Long chain fatty acids of the n-7 and n-9 series are on the other hand often defined as being “non-essential” as they can biosynthetized de novo.

Mixture of such compounds are also comprised within the scope of the invention and/or the term.

The term “fatty acid derivative” as used herein refers to a compound comprising a fatty acid, other than a phospholipid, and in particular to a free fatty acid, and/or a monoacylglycerol (hereinafter MAG), and/or a diacylglycerol (hereinafter DAG), and/or a triacylgylcerol (hereinafter TAG) and/or a cholesterol ester. More particularly the term refers to a MAG, DAG, TAG and/or a cholesterol ester. Even more particularly the term refers to a TAG.

Mixture of such compounds are also comprised within the scope of the invention and/or the term.

The term “MAG” as used herein refers to a glycerol molecule in which one of the OH groups has formed an ester bond with a fatty acid. In particular the term “MAG” as used herein refers to a compound of formula (X)

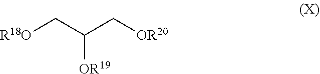

Wherein,

two of R¹⁸ R¹⁹ or R²⁰ are H and wherein one of R¹⁸ R¹⁹ or R²⁰ is a C4 to C44 saturated or unsaturated acyl group.

Mixture of such compounds are also comprised within the scope of the invention and/or the term.

The term “DAG” as used herein refers to glycerol molecule in which two of the OH groups have formed an ester bond with two fatty acids. In particular the term “DAG” as used herein refers to a compound of formula (X)

Wherein, one of R¹⁸ R¹⁹ or R²⁰ are H and wherein two of R¹⁸ R19 or R²⁰ are C4 to C44 saturated or unsaturated acyl groups. The two C4 to C44 saturated or unsaturated acyl groups may be the same or different.

Mixture of such compounds are also comprised within the scope of the invention and/or the term.

The term “TAG” as used herein refers to glycerol molecule in which three of the OH groups have formed an ester bond with three fatty acids. In particular the term “TAG” as used herein refers to a compound of formula (X)

Wherein,

Wherein all R¹⁸ R¹⁹ or R²⁰ are C4 to C44 saturated or unsaturated acyl groups. The three C4 to C44 saturated or unsaturated acyl groups may all be the same, all different, or two may be the same and one different.

Mixture of such compounds are also comprised within the scope of the invention and/or the term.

The term “cholesterol ester” as used herein refers to a compound of formula (XI)

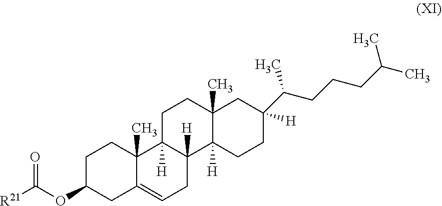

Wherein,

R²¹ is a C2 to C43 branched or unbranched acyclic alky, or acyclic alkenyl group.

Mixture of such compounds are also comprised within the scope of the invention and/or the term.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995;125:1401-12).

The term “vitamin” as used herein refers to any vitamin. Non limiting examples of vitamins include: vitamin A, vitamin B1, vitamin B2, vitamin B6, vitamin K, vitamin C, vitamin D, niacin, biotin, pantothenic acid, folic acid, vitamin B12, and combinations thereof.

Within the context of the present invention, the term “folic acid” is to be intended as identifying all the folic acid present in the nutritional compositions, for example synthetic nutritional compositions, of the invention either as such or in the form of one physiologically acceptable salt thereof (folate) and mixtures thereof.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

The terms “in particular” or “more particularly” as used herein should not be considered limiting but should be interpreted as being synonymous with “for example” or “especially”.

The invention will now be described in further details. It is noted that the various aspects, features, examples and embodiments described in the present application may be compatible and/or combined together.

### Experimental Section

Embodiments

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

Preterm Infant Formula

In one embodiment, nutritional compositions according to the present invention is a pre-term formula.

In one embodiment, the pre-term formula according to the present invention comprises MCFA in an amount of up to 40% by weight of the total content of lipid.

In an embodiment of the invention, the preterm formula comprises at least 20% MCT by weight of the total lipid content, such as at least 25%, preferably at least 30%, such as at least 35%, even more preferably 40% by weight of the total lipid content.

In one embodiment, the preterm formula according to the present invention comprises MCFA derivatives in amount ranging from 0.1 to 25% w/w, for example in an amount ranging from 0.5 to 20% w/w, for example in an amount ranging from 1 to 15% w/w of dry powder.

In another embodiment, the liquid preterm formula according to the present invention comprises MCFA derivatives in amount ranging from 0.01 to 4 g/100 mL of liquid formula, for example in an amount ranging from 0.05 to 3 g/100 mL, for example in an amount ranging from 0.1 to 3.5 g/100 mL.

In another embodiment, the preterm formula according to the present invention comprises MCFA derivatives in amount ranging from 0.01 to 5 g/100 Kcal of formula, for example in an amount ranging from 0.05 to 4 g/100 Kcal, for example in an amount ranging from 0.1 to 3 g/100 Kcal.

In one embodiment, the preterm formula according to the present invention comprises fatty acid derivatives in amount ranging from 10 to 40% w/w, MCFA derivatives in amount ranging from 0.1 to 25% w/w, 5 to 50% w/w protein and 10 to 80% w/w carbohydrates.

Human Milk Fortifier

In one embodiment, the nutritional compositions according to the present invention is a human milk fortifier.

In one embodiment, the human milk fortifier according to the present invention comprises MCFA derivatives in amount ranging from 2 to 40% w/w, for example in an amount ranging from 5 to 30% w/w, for example in an amount ranging from 5 to 20% w/w for example in an amount ranging from 7 to 18% w/w.

In another embodiment, the human milk fortifier according to the present invention comprises MCFA derivatives in amount ranging from 0.08 to 1.6 g/100 mL of HMF reconstituted in human breast milk, for example in an amount ranging from 0.2 to 1.2 g/100 mL, for example in an amount ranging from 0.25 to 0.75 g /100 mL.

In another embodiment, the human milk fortifier according to the present invention comprises MCFA derivatives in amount ranging from 2 to 10 g/100 Kcal of HMF, for example in an amount ranging from 1.2 to 7.5 g/100 Kcal, for example in an amount ranging from 1.75 to 4.5 g/100 Kcal.

In another embodiment, the human milk fortifier according to the present invention comprises MCFA derivatives in amount ranging from 0.05 to 2.5 g/100 Kcal of HMF reconstituted in human breast milk, for example in an amount ranging from 0.2 to 2.0 g/100 Kcal, for example in an amount ranging from 0.5 to 1.5 g /100 Kcal.

In one embodiment, the human milk fortifier according to the present invention comprises MCFA derivatives in amount ranging from 40 to 80% w/w of total fatty acid derivatives, for example in an amount ranging from 50 to 75% w/w, for example in an amount ranging from 55 to 70% w/w.

In another embodiment, the human milk fortifier according to the present invention comprises MCFA derivatives in amount ranging from 5 to 40% w/w of total fatty acid derivatives/100 mL of HMF reconstituted in human breast milk, for example in an amount ranging from 10 to 20% w/w of total fatty acid derivatives/100 mL of HMF reconstituted in human breast milk.

In another embodiment, the human milk fortifier according to the present invention comprises MCFA derivatives in amount ranging from 5 to 40% w/w of total fatty acid derivatives/100 Kcal of HMF reconstituted in human breast milk, for example in an amount ranging from 10 to 20% w/w of total fatty acid derivatives/100 Kcal of HMF reconstituted in human breast milk.

In one embodiment, the human milk fortifier according to the present invention comprises 5 to 40% w/w fatty acid derivatives, wherein 40 to 80% w/w are constituted by MCFA derivatives.

In one embodiment, the human milk fortifier according to the present invention comprises 5 to 30% w/w fatty acid derivatives, wherein 50 to 75% w/w are constituted by MCFA derivatives, 20 to 50% w/w protein and 15 to 40% w/w carbohydrates.

### Other Ingredients

The nutritional composition, according to the present invention, for example the synthetic nutritional composition, can besides from comprising MCFA derivatives comprise other nutrients, such as e.g. lipids (including fatty acid derivatives), proteins, carbohydrates, vitamins, minerals, probiotics, or prebiotics.

### Lipids

In the context of the present invention, the term “lipid” refers to one or more lipids and may be any free fatty acid or ester of fatty acids that are suitable for being fed to an infant. Lipid includes for example monoglycerides, diglycerides, triglycerides, phospholipids, cholesterol, free fatty acids, derivatives of fatty acids and combinations thereof.

The lipids used to prepare the fortifier can be naturally liquid or solid at room temperature. In some particular embodiments at least a part of the lipids used to prepare the fortifier are naturally liquid at room temperature.

In an embodiment of the present invention, the nutritional composition, for example the HMF according to the invention, comprises lipid in an amount above 25% of the caloric content.

In another embodiment, the nutritional composition, for example the HMF according to the invention, comprises lipid in an amount above 75% of the caloric content. In some embodiments of the invention, lipids are present in the nutritional composition, for example the HMF, in an amount of at least 30% of the caloric content, such as at least 35% of the caloric content.

In an embodiment of the invention, the lipids are selected from the group of monoglycerides, diglycerides, triglycerides, phospholipids, cholesterol, free fatty acids, derivatives of fatty acids and combinations thereof.

In a particular embodiment of the invention, the lipids are selected from the group of arachidonic acid, docosahexaenoic acid, eicosapentaenoic acid, linoleic acid, a-linolenic acid, milk fat, structured lipids phospholipid, and combinations thereof. Structured lipids may be monoglycerides, diglycerides, triglycerides, cholesterol, palmitic acid esterified in the sn-2 position or interesterified palm stearin.

Lipids may be derived from various sources. The lipid source may be any lipid or fat source which is suitable for use in nutritional compositions, to be fed to infants, for example some vegetable or animal fats or oils.

In an embodiment of the invention, the lipid is provided from oils or fats.

Preferred lipid sources include coconut oil, soy oil, corn oil, olive oil, safflower oil, sunflower oil, palm oil, palm kernel oil, low erucic rapeseed oil (canola oil), marine oil, cottonseed oil, soy lecithin's, palm oil, milk fat, structured lipids, egg-derived oils, fungal oils, algal oils and combinations thereof. Particularly preferred oils are canola oils, soy lecithin, palm olein, and sunflower oil.

Dietary lipids are essential for an infant since they provide the infant with much of his energy needs, such as the essential polyunsaturated fatty acids and lipid soluble vitamins. The amount and composition of dietary lipids affect both the growth pattern and the body composition of the infant.

In an embodiment of the invention, the lipid comprises one or more polyunsaturated fatty acid, preferably long chained polyunsaturated fatty acids.

The polyunsaturated fatty acids, and in particular the long chain ones are important for the cell membrane function and the development of the brain and visual system in infants. Further, the long chain polyunsaturated fatty acids are important in the formation of bioactive eicosanoids. Brain grey matter and the retina are complex neural functions related to energy supply and the composition of dietary fatty acids.

In a particular embodiment of the invention, the composition comprises arachidonic acid, docosahexaenoic acid, or a combination thereof as the lipid component. The arachidonic acid and docosahexaenoic acid may be alone or in combination with other lipids, such as linoleic acid and/or a-linolenic acid.

In one embodiment, the content of arachidonic acid in the nutritional composition according to the invention, for example a HMF, is at least 0.005% w/w, such as at least 0.0075%, for example at least 0.01% w/w.

In one embodiment, the content of arachidonic acid in the nutritional composition of the invention, for example a preterm formula, ranges between 0.001% w/w to 1% w/w, for example from 0.01% w/w to 0.5% w/w.

In one embodiment, the content of arachidonic acid in the HMF according to the present invention is at least 0.2% by weight of the total lipid content, such as at least 0.30%, in particular at least 0.38%, even more preferably at least 0.65%, such as 0.70% by weight of total lipid content.

In another embodiment the HMF comprises arachidonic acid in an amount of up to 2.5% by weight on the total lipid content, such as at in the range of 0.2 to 2.0%, preferably from 0.3 to 1.5%, such as from 0.35 to 1.2%, even more preferably from 0.4 to 0.9% by weight of the total lipid content.

In one embodiment, the content of docosahexaenoic acid in the nutritional composition of the invention, for example a preterm formula, ranges between 0.001% w/w to 1% w/w, for example from 0.01% w/w to 0.5% w/w.

In one embodiment, the content of docosahexaenoic acid in the nutritional composition according to the invention, for example a HMF, is at least 0.05% w/w, such as at least 0.075% w/w, for example at least 0.1% w/w.

In one embodiment, the content of docosahexaenoic acid in the HMF according to the present invention is ranging from 0.05% to 5% w/w, such as from 0.075% to 3% w/w, for example from 0.1% to 2% w/w.

In one embodiment, the content of docosahexaenoic acid in the HMF according to the present invention is preferably at least 0.05% by weight of the total lipid content, such as at least 0.1%, for example at least 0.15%, such as 0.5% by weight of total lipid content.

In another specific embodiment the composition comprises docosahexaenoic acid in an amount of up to 3.0% by weight on the total lipid content, such as from 0.0.5% to 2.5%, preferably from 0.1 to 2.0%, such as from 0.15 to 1.50%by weight of the total lipid content.

In on embodiment, if the nutritional composition according to the present invention, comprises fatty acid derivatives comprising ARA and DHA, said ingredients may for example be comprised in the composition of the invention in amounts resulting in a weight ratio of DHA:ARA in the range of 4:1 to 1:4, for example 3:1 to 1:3, for example 2:1 to 1:2, for example 1.5:1 to 1:1.5, in particular 1.1:1 to 1:1.1.

Docosahexaenoic (DHA) and arachidonic acid (ARA) are both known to provide beneficial effects in infants, such as enhancing brain and vision development. DHA and ARA are therefore necessary for infants, both preterm and term infants, but in particular for a preterm infant.

Non-limiting examples of suitable sources of ARA and DHA include marine oil, egg-derived oils, fungal oil, algal oil, and combinations thereof.

In still another embodiment of the invention, the nutritional composition according to the invention, for example a synthetic nutritional composition, comprises linoleic acid, α-linolenic acid or a combination thereof as lipid.

In a specific embodiment of the invention, the nutritional composition of the invention, for example a human milk fortifier, comprises linoleic acid in an amount ranging from 0.1% w/w to 5% w/w of dry composition.

In another embodiment of the invention, the nutritional composition of the invention, for example a preterm powder formula, comprises linoleic acid in an amount ranging from 0.5 to 10% w/w of dry composition.

In another embodiment of the invention, the nutritional composition of the invention, for example a preterm liquid formula, comprises linoleic acid in an amount ranging from 0.05 to 5 g/100 mL of formula.

In a specific embodiment of the invention, the nutritional composition of the invention, for example a human milk fortifier, comprises α-linolenic acid in an amount ranging from 0.1% w/w to 3% w/w of dry composition.

In another embodiment of the invention, the nutritional composition of the invention, for example a preterm powder formula, comprises a-linolenic acid in an amount ranging from 0.01 to 5% w/w of dry composition.

In another embodiment of the invention, the nutritional composition of the invention, for example a preterm liquid formula, comprises a-linolenic acid in an amount ranging from 0.01 to 2 g/100 mL of formula.

The lipid may also be eicosapentaenoic acid (20:5n-3).

In a specific embodiment of the invention, the nutritional composition of the invention, for example a human milk fortifier, comprises eicosapentaenoic acid in an amount ranging from 0.01% w/w to 5% w/w of dry composition.

In another embodiment of the invention, the nutritional composition of the invention, for example a preterm powder formula, comprises eicosapentaenoic acid in an amount ranging from 0.01 to 5% w/w of dry composition.

In another embodiment of the invention, the nutritional composition of the invention, for example a preterm liquid formula, comprises eicosapentaenoic acid in an amount ranging from 0.05 to 20 mg/100 mL of formula.

In an embodiment of the invention, the lipid comprises one or more of phospholipids.

In one embodiment, the content of phospholipid in the composition according to the present invention, for example a human milk fortifier, is preferably from 0.5 to 20% by weight of the total lipid content, such as from 0.8 to 15%, even more preferably from 1.0 to 10%, such as from 1.5 to 8% by weight of the total content of lipid.

In one embodiment, phospholipids may be phosphatidylcholine, phosphatidylserine, phosphatidylinositol and/or sphingomyelin, in particular sphingomyelin. However in a particular embodiment of the invention, the composition according to the present invention does not comprise any phospholipids.

### Additional Ingredients

The compositions of the invention can also comprise any other ingredients or excipients known to be employed in the type of composition in question e.g. infant formula, preterm formula and/or human milk fortifiers.

Non limiting examples of such ingredients include: proteins, amino acids, carbohydrates, oligosaccharides, lipids, prebiotics or probiotics, nucleotides, nucleosides, other vitamins, minerals and other micronutrients.

### Vitamins

The composition according to the present invention may further comprise one or more vitamin. The presence and amounts of specific minerals and other vitamins will vary depending on the intended population.

In one embodiment, vitamins may be folic acid, vitamin B12 and vitamin B6, in particular folic acid and vitamin B12, in particular folic acid.

In one embodiment of the invention, the composition comprises one or more vitamin which is lipid-soluble, for example one or more of vitamin A, vitamin D, vitamin E and vitamin K.

Vitamin D is important for supporting a large number of physiological processes such as neuromuscular function and bone mineralisation. The preferred amount of vitamin D given to an infant in the first months of life is 800-1000 IU per day, i.e. 20-25 μg per day.

Only small amounts of vitamin D are transported to the breast milk. Thus, human breast milk contains low amounts of vitamin D. An infant who is breast fed therefore will need an additional supply of vitamin D. There is therefore a need for a nutritional composition, for example a synthetic nutritional composition, to supply energy to an infant which also contributes to the recommended intakes of vitamin D.

An infant is normally fed 5-8 times a day, and the amount of vitamin per serving should therefore not exceed 5.0 μg vitamin D, preferably the amount per serving should be 3-4 μg vitamin D.

In one embodiment, the amount of vitamin Din the nutritional composition, in particular a human milk fortifier, is thus preferably from 75 to 125 μg per 100 g of the total composition, such as from 80 to 120 μg per 100 g of the total composition, even more preferably from 85 to 110 μg per 100 g of the total composition.

In an embodiment of the invention, the composition comprises from 0.5 to 10.0 μg vitamin D per 100 kcal of the composition, such as from 1.0 to 8.0 μg vitamin D per 100 kcal, preferably from 2.0 to 7.0 μg vitamin D per 100 kcal, even more preferably from 3.5 to 5.5 μg vitamin D per kcal of the composition.

Vitamin K is important to help blood to clot. The human breast milk contains low amounts of vitamin K and the infants immature intestinal tract may not produce enough vitamin K to meet the infants own needs.

In one embodiment, the amount of vitamin K in the nutritional composition according to the present invention, for example a human milk fortifier, is preferably from 50 to 400 μg per 100 g of the total composition, such as from 100 to 300 μg per 100 g of the total composition, preferably 200 μg per 100 g of the total composition.

In an embodiment of the invention, the nutritional composition, comprises from 1 to 30 μg vitamin K per 100 kcal, such as form 5 to 20 μg vitamin K per 100 kcal, preferably from 7 to 15 μg vitamin K per 100 kcal, even more preferably from 8 to 12 μg vitamin K per 100 kcal.

Vitamin A prevents infections, while vitamin E protects the body from harmful substances and serves as an antioxidant. The daily intake of vitamin A in an infant is preferably from 400 to 1000 μg/kg/day.

Thus, in an embodiment of the invention, the nutritional composition of the invention, for example a human milk fortifier, comprises from 1 to 30 mg vitamin A per 100 g of the total composition, such as from 5 to 20 mg per 100 g of the total composition, preferably from 8 to 15 mg per 100 g of the total composition.

In an embodiment of the invention, the composition comprises from 0.1 to 3.0 mg vitamin A per 100 kcal, such as from 0.2 to 2.0 mg vitamin A per 100 kcal, preferably from 0.3 to 1.2 mg vitamin A per 100 kcal, even more preferably from 0.4 to 0.8 mg vitamin A per 100 kcal.

The daily intake of vitamin E in an infant is preferably 2.2 to 11 mg per day. Thus, in an embodiment of the invention, the nutritional composition of the invention, in particular a human milk fortifier, comprises from 50 to 200 mg vitamin E per 100 g of the total composition, such as from 75 to 150 mg vitamin E per 100 g of the total composition, preferably from 85 to 115 mg vitamin E per 100 g of the total composition.

In an embodiment of the invention, the composition comprises from 1 to 10.0 mg vitamin E per 100 kcal, such as from 2 to 8.0 mg vitamin E per 100 kcal, preferably from 3 to 7 mg vitamin E per 100 kcal, even more preferably from 4 to 6 mg vitamin E per 100 kcal.

Minerals:

In an embodiment of the invention, the composition further comprises one or more mineral.

Examples of minerals are sodium, potassium, chloride, calcium, phosphate, magnesium, iron, zinc, copper, selenium, manganese, fluoride, iodine, chromium, or molybdenum. The minerals are usually added in salt form.

The minerals may be added alone or in combination.

In on embodiment, minerals may be iron, zinc, calcium, phosphorus, copper, and magnesium, in particular iron.

In a specific embodiment of the invention, the mineral is calcium.

Protein:

In another embodiment of the invention the composition further comprises a protein source. The composition may comprise one or more protein.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. The proteins can be at least partially hydrolyzed in order to enhancement of oral tolerance to allergens, especially food allergens. In that case the composition is a hypoallergenic composition.

In one embodiment, the nutritional composition according to the invention may be cow's milk whey based infant formula. The formula may also be a hypoallergenic (HA) formula in which the cow milk proteins are (partially or extensively) hydrolysed. The formula may also be based on soy milk or a non-allergenic formula, for example one based on free amino acids.

In one embodiment, the nutritional composition cow milk proteins which are partially hydrolysed. In a further embodiment, the nutritional composition comprises cow milk proteins which are partially hydrolysed in an amount ranging from 30 to 40 w/w of the nutritional composition.

In an embodiment of the invention, the nutritional composition, for example a human milk fortifier, comprises up to 55% protein of the caloric content, for example up to 50%. In a preferred embodiment of the invention, the composition comprises up to 45% protein, such as up to 40% protein, or up to 35% protein, based on the caloric content.

In another embodiment of the invention, the composition is free of protein. By “free” is hereby meant that the composition may comprise traceable amounts of protein, such as less than 1% protein.

In the context of the present invention, the term “protein” refers to both proteins derived from a source of protein, to peptides and to free amino acids in general. There can be one or several proteins.

In an embodiment of the invention, protein, if present, is made of whey proteins.

In another embodiment of the invention, the protein, if present, comprises lactoferrin. The protein(s) in the protein source may be intact or hydrolysed or a combination of intact and hydrolysed proteins.

The term “intact” means in the context of the present invention proteins where the molecular structure of the protein(s) is not altered according to conventionally meaning of intact proteins. By the term “intact” is meant the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component peptides or amino acids.

The proteins may either be fully or partially hydrolysed. In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

Hydrolysation of proteins may be achieved by many means, for example by prolonged boiling in a strong acid or a strong base or by using an enzyme such as the pancreatic protease enzyme to stimulate the naturally occurring hydrolytic process.

The protein(s) according to the present invention may also be derived from free amino acids, or a combination of free amino acids and a source of protein, such as whey, lactoferrin and casein.

The whey protein may be a whey protein isolate, acid whey, sweet whey or sweet whey from which the caseino-glycomacropeptide has been removed (modified sweet whey). Preferably, however, the whey protein is modified sweet whey.

Carbohydrates:

The composition according to the present invention can also contain a carbohydrate source, preferably as prebiotics, or in addition to prebiotics. Any carbohydrate source conventionally found in infant formulae such as lactose, saccharose, maltodextrin, starch and mixtures thereof may be used although the preferred source of carbohydrates is lactose.

The composition may comprise one or more carbohydrate.

In an embodiment of the invention, the nutritional composition, for example a human milk fortifier, comprises up to 40% carbohydrate of the caloric content. In a particular embodiment of the invention, the composition comprises up to 35% carbohydrate, such as up to 300% carbohydrate, based on the caloric content.

In another embodiment of the invention, the composition is free of carbohydrate. By “free” it is hereby meant that the composition may comprise traceable amounts of carbohydrates, such as less than 1% carbohydrate.

Non limiting examples of carbohydrates include lactose, saccharose, maltodexirin, starch, and combinations thereof.

### Probiotics

The nutritional composition according to the present invention, for example the synthetic nutritional composition, may optionally comprise other compounds which may have a beneficial effect such as probiotics (like probiotic bacteria) in the amounts customarily found in nutritional compositions to be fed to infants.

Strains of Lactobacillus are the most common microbes employed as probiotics. However, other probiotic strains than Lactobacillus may be used in the present nutritional composition, for example the synthetic nutritional composition, for example Bifidobacterium and certain yeasts and bacilli.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. Probiotic bacteria are bacteria which have a beneficial effect on the intestinal system of humans and other animals.

In some specific embodiments, it is particularly Bifidobacteria and/or Lactobacilli.

A probiotic is a microbial cell preparation or components of microbial cells with a beneficial effect on the health or well-being of the host.

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

In an embodiment of the invention, the infant formula further includes a probiotic strain such as a probiotic bacterial strain in an amount of from 10⁶ to 10¹¹ cfu/g of composition (dry weight).

In an embodiment of the invention, the composition further comprises one or more probiotic.

### Prebiotics

In one embodiment, the nutritional composition according to the present invention may optionally comprise one or more prebiotic. In one embodiment, the synthetic nutritional composition according to the present invention comprises one or more prebiotic.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416 which is incorporated herein by reference.

Emulsifiers

If necessary, the nutritional composition according to present invention, for example the synthetic nutritional composition, may comprise emulsifiers and/or stabilizers such as lecithin, citric esters of mono- and diglycerides, monoglycerides, diglycerides and the like. This is especially the case if the composition is provided as a combination of oils and an aqueous liquid, e.g. as an emulsion.

### Additional Ingredients

The nutritional composition of the present invention, for example the synthetic nutritional composition, may also optionally comprise other substances which may have a beneficial effect such as nucleotides, nucleosides, and the like in the amount customarily found in nutritional compositions to be fed to infants.

Other optional ingredients may be ones normally known for use on food and nutritional products, in particular infant formulas or infant formula fortifiers, provided that such optional materials are compatible with the essential components described herein, are safe and effective for their intended se, and do not otherwise unduly impair product performance.

Non-limiting examples of such optional ingredients include preservatives, anti-oxidants, buffers, colorants, flavours, thickening agents, stabilizers, and other excipients or processing aids.

Preparation

The composition according to the present invention may be prepared in any suitable manner. For example, a composition may be prepared by blending together the ingredients, such as lipid, protein and/or carbohydrate in appropriate proportions. If used, emulsifiers may be included in the blend at this stage. The vitamins and minerals may be added at this stage but are usually added later to avoid thermal degradation. Any lipophilic vitamins, such as vitamin A, D, E and K, and emulsifiers may be dissolved into the fat source prior to blending. Water, preferably water which has been subjected to reverse osmosis, may then be mixed in to a liquid mixture.

The mixture may then be thermally treated to reduce bacterial loads. Any heat sensitive components, such as vitamins and minerals may be added after heat treatment.

### EXAMPLE 1

An example of the composition of a fortifier according to the invention is given in the below table 1.

TABLE 1

| Nutrient | per 100 g |
|---|---|
| Energy (kcal) | 435.5 |
| Total solids | 97.0 |
| Water | 3.0 |
| Protein (g) | 35.5 |
| Fat (g) | 18.1 |
| Saturated Fatty acids (g) | 12.20 |
| Medium chain tryglycerides (g) | 12.50 |
| DHA (mg) | 157.00 |
| ALA (mg) | 417.00 |
| LA (mg) | 958.00 |
| Carbohydrates (g) | 32.40 |
| Ash (g) | 11 |
| Minerals (g) | 6.906 |
| Vitamin A (μg RE) | 8875.00 |
| Vitamin D (μg D) | 94.00 |
| Vitamin E (mg TE) | 100.00 |
| Other ingredients (amino acids, | Summing |
| vitamins, etc) | up to 100 g |

### Example 2

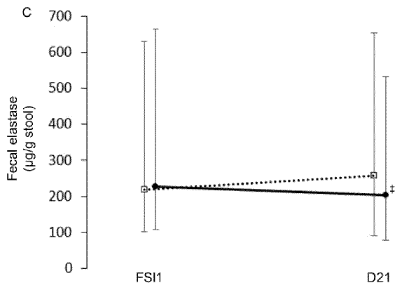

The present example illustrates the effects of a human milk fortifier according to the Invention for reducing the metabolic stress (FIG. 1).

Results are hereby presented from a randomized, controlled clinical trial conducted in neonatal intensive care units at 11 hospitals located in 5 countries in Europe.

Methods

Clinically stable preterm infants with gestational age ≤32 weeks or birthweight ≤1500 g and born to mothers choosing to provide breastmilk were eligible for enrollment. Infants tolerating ≥100 mL/kg/day of HM for ≥24 hours were randomized to receive either nHMF or cHMF (described in Table 2) until study day 21 (D21). Fortifiers were fed starting at half-strength (Fortification Strength Increase day 1; FSI1) and advanced per hospital practice, with full-strength fortification occurring once infants could maintain intakes of 150-180 mL/kg/day (i.e. full enteral feeds; study day 1 [D1]).

TABLE 2
Nutritional composition of the control (cHMF) and new
human milk fortifier (nHMF) used in the present study

| Nutrients (per 100 g of powder) | cHMF | nHMF |
|---|---|---|
| Protein (g) | 20.00 | 35.50 |
| Carbohydrates (g) | 66.00 | 32.40 |
| Lipid content (g) | 0.38 | 18.10 |
| Saturated fatty acids (g) | — | 12.20 |
| Medium chain fatty acids (MCFA, g) | — | 12.50 |
| Linoleic acid (LA, mg) | — | 958.00 |
| α-Linolenic acid (ALA, mg) | — | 417.00 |
| Docosahexaenoic acid (DHA, mg) | — | 157.00 |
| Vitamin A (μg RE) | 3000.00 | 8875.00 |
| Vitamin D (μg D) | 50.00 | 94.00 |
| Vitamin E (mg TE) | 40.00 | 100.00 |

Stool samples were collected at FSI1+1 and D21±1 and analyzed for the maturity of gut function (fecal elastase-1). Approximately 5-8 g of stool was collected from each infant within 2 hours of the bowel movement. Samples were stored frozen (−20° C.) and shipped for analysis on dry ice. If stool sample quantity was insufficient (<5.5 g), a second collection was made from a later bowel movement on the same or following day. Concentrations of elastase-1 was assessed by enzyme-linked immunosorbent assays (ScheBo Pancreatic Elastase 1, ScheBo Biotech AG, Giessen, Germany; Euroimmun Analyzer A1, Euroimmun, Lübeck, Germany) following sample treatment with Roche Diagnostics fecal extraction device (Mannheim, Germany). Analyses were completed in a central laboratory (Rothen Medizinische Laboratorien AG, Basel, Switzerland).

FSI11 values were log-transformed and groups compared using t-tests computed with the Satterthwaite method (24); D21. values were log-transformed and analyzed using ANCOVA adjusting for FSI1 value of the relevant parameter, sex, and center (random effect). Changes from FSI1 to D21. were analyzed using ANCOVA adjusting for postmenstrual age at D1 (i.e., start of full enteral feeding/full fortification), weight at D1, FSI1 value of the relevant parameter, sex, and center (random effect).

The study was reviewed and approved by an Institutional Review Board/Independent Ethics Committee at each hospital and the parent or legal representative of each participant provided written informed consent prior their enrollment.

Results

A total of 153 infants were enrolled and randomized to either nHMF (n=77) or cHMF (n=76). No imbalance was observed between the 2 groups with regard to infant characteristics. The subject population providing data for the stool analyses (ranging from 15 to 130) was smaller than the full sample size due to difficulty in obtaining sufficient stool quantity for all subjects.

There were no significant differences in fecal biomarker at FSI1. As shown in the FIG. 1, the geometric mean concentration of fecal elastase-1 was significantly lower in the nHMF group compared to the cHMF group at D21 (P=0.016). In addition, the increase from FSI1 to D21 was significantly less in the nHMF group compared to the cHMF group (P=0.004).

A significant difference between groups was also observed in fecal elastase-1 concentration, with a lower mean value at D21 in the nHMF group vs. cHMF. Elastase-1 secretion in stool is considered to be a marker of pancreatic insufficiency (with values≥200 μg/g stool indicating non-impaired exocrine pancreatric function).

Accordingly, it results from the present study that pre-term infants fed with human milk fortified with a human milk fortifier according to the present invention presented levels of faecal elastase which were normalized (i.e. higher than 200 μg/g stool) and lower than the control groups fed with human milk fortified with a standard fortifier. These findings indicate that the compositions of the invention contribute to a positive effect on homeostasis and may dampen a stress on metabolic functions for the infant, in particular an infant born preterm or with low-birth weight (LBW) or who experienced intra-uterine growth retardation (IUGR) or small for gestational age (SGA), who is fed with the composition.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A method for reducing metabolic stress in a subject in need of same comprising administering a nutritional composition comprising one or more medium chain fatty acid (MCFA) derivative.

2. Method according to claim 1 wherein the subject is a human infant or child.

3. Method according to claim 1 wherein one or more MCFA derivative are provided in the form of TAGs.

4. Method according to claim 1 for the prevention and/or treatment of conditions and/or diseases associated to the metabolic disorders and/or imbalances in an infant.

5. Method according to claim 4 wherein conditions and/or diseases associated to the metabolic disorders and/or imbalances in an infant are selected in the group consisting of: neurological, growth and/or gut retarded development or abnormalities, hypoglycemia, hyperglycemia, hyperinsulinemia, hypertriglyceridemia.

6. Method according to claim 5 wherein the infant is an infant who was born preterm or with low-birth weight (LBW) or who experienced intra-uterine growth retardation (IUGR) or small for gestational age (SGA).

7. Method according to claim 1 wherein the composition is a human milk fortifier.

8. Method according to claim 7 wherein the composition comprises 5 to 40% w/w fatty acid derivatives.

9. Method according to claim 1 wherein the composition comprises one or more of vitamin A, vitamin D, vitamin E and vitamin K.

10. Method according to claim 1 wherein the composition comprises 30 to 40% w/w partially hydrolyzed cow milk proteins.

11. Method according to claim 1 wherein the composition comprises 5 to 30% w/w fatty acid derivatives, wherein 50 to 75% w/w are constituted by MCFA derivatives, 20 to 50% w/w protein and 15 to 40% w/w carbohydrates and 0.05% to 5% w/w, and one or more of vitamin A, vitamin D, vitamin E and vitamin K, and 30 to 40% w/w partially hydrolyzed cow milk proteins.

12. Method according to claim 1 wherein the composition is a synthetic nutritional composition.

13. (canceled)

14. Method for reducing metabolic stress in an infant in need thereof comprising administering to such infant a nutritional composition comprising one or more medium chain fatty acids (MCFA) derivative.

15. (canceled)
